(19) United States
(12) Patent Application Publication
(10) Pub. No.: US 2018/0256485 A1
(43) Pub. Date: Sep. 13, 2018

(54) SERUM FOR THE REDUCTION OF WRINKLES AND OTHER SKIN ISSUES

(71) Applicant: Karen Drake, Bel Air, CA (US)

(72) Inventor: Karen Drake, Bel Air, CA (US)

(21) Appl. No.: 15/455,660

(22) Filed: Mar. 10, 2017

(51) Int. Cl. A61K 8/97; A61Q 19/08; A61K 8/25; A61K 8/34; A61K 8/73; A61K 8/26; A61K 8/64

(52) U.S. Cl. CPC A61K 8/97; A61Q 19/08; A61K 8/25; A61K 8/345; A61K 2800/48; A61K 8/26; A61K 8/64; A61K 8/34; A61K 8/73

(57) ABSTRACT

A serum for improving the youthful appearance of skin by reducing the appearance of fine lines and wrinkles may include sodium silicate; glycerin extract; pullulan; propylene glycol; magnesium aluminum silicate; and xanthan gum. The serum may also include water; rosa damascena flower extract; acetyl hexapeptide—8, caprylyl glycol, water extract; cucumis sativus fruit extract; glycyrrhiza glabra root extract; phenoxyethanol; and ethylhexylglycerin. The serum may be made by a specific process to ensure desired properties.

### BACKGROUND

The embodiments herein relate generally to skin treatment compositions, and more particularly, to serum for the reduction in the appearance of wrinkles and other skin issues or anomalies.

Aging is brought about by a cycle of various biochemical processes in the body that affect the body both internally and externally. These biochemical processes cause the body to degenerate over a period of time, impacting the health, fitness, and beauty of an aging individual.

The prevalence of anti-aging products and services is becoming more prominent now as the aging Baby Boomers have spurred a demand for various methods that can either reverse or slow the signs of aging. Estimates indicate that over the next 18 years, individuals from this generation will attain the age of 65 at the rate of 8,000 per day.

Anti-aging products have gained wide acceptance, as they can often be purchased off the shelf or with a prescription and are relatively affordable. The anti-aging services market is further categorized into the cosmetic surgery segment and the non-invasive treatment segment, which are dependent on plastic surgeons, qualified medical practitioners, and certified beauticians.

Currently, the challenges for many looking to gain a more youthful, fresh appearance is either (a) the cost of surgery, or (b) the concerns about going through such an invasive process in the pursuit of youth. However, for some individuals, there is an additional challenge. Particularly, available serums and creams aimed at reducing wrinkles and improving the skin's youthful appearance tend to be incompatible with certain skin types and colors. Specifically, when it comes to women of color and Asian skin types, existing serums and creams do not dry completely transparently. Rather, the existing serums and creams, when dry, tend to leave a light-colored demarcation that is visible on darker skin types.

Therefore, what is needed is a skin treatment that improves the youthful appearance of the skin while simultaneously drying on all skin types and colors without leaving a visible demarcation. In other words, what is needed is a serum that will dry clear on all skin types while simultaneously improving the appearance of the skin by, for example, reducing or removing the presence of wrinkles, which may be accomplished by instantly or almost instantly tightening and lifting the skin, causing wrinkles, eye bags, and puffiness to flatten out.

### SUMMARY

Some embodiments of the present disclosure include a serum for improving the youthful appearance of skin by reducing the appearance of fine lines and wrinkles may include sodium silicate; glycerin extract; pullulan; propylene glycol; magnesium aluminum silicate; and xanthan gum. The serum may also include water; rosa damascena flower extract; acetyl hexapeptide—8, caprylyl glycol, water extract; cucumis sativus fruit extract; glycyrrhiza glabra root extract; phenoxyethanol; and ethylhexylglycerin. The serum may be made by a specific process to ensure desired properties. Specifically, the serum of the present disclosure may be suitable for use on all skin types and colors without leaving a white demarcation upon drying.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The serum of the present disclosure may be used to improve the youthful appearance of the skin by, for example, removing or reducing the presence of wrinkles and may comprise the following elements. This list of possible constituent elements is intended to be exemplary only, and it is not intended that this list be used to limit the serum of the present application to just these elements. Persons having ordinary skill in the art relevant to the present disclosure may understand there to be equivalent elements that may be substituted within the present disclosure without changing the essential function or operation of the serum.

1. Water

2. Sodium Silicate

3. Rose Flower Extract

4. Propylene Glycol

5. Magnesium Aluminum Silicate

6. Glycerin

7. Pullulan

8. Acetyl Hexapeptide—8, Caprylyl Glycol, Water Extract

9. Cucumber Fruit Extract

10. Licorice Root Extract

11. Xanthan Gum

12. Phenoxyethanol

13. Ethylhexylglycerin

The various elements of the composition of the present disclosure may be related in the following exemplary fashion. It is not intended to limit the scope or nature of the relationships between the various elements and the following examples are presented as illustrative examples only.

By way of example, some embodiments of the present disclosure include a serum for improving the youthful appearance of skin, the serum comprising a solvent, a tightening agent, a botanical, a fragrance, a humectant, a film former, a peptide compound, a thickener, and a preservative, wherein the serum may provide lifting and tightening effects while avoiding a whitening effect.

Suitable solvents for the serum of the present disclosure include water and propylene glycol. Suitable tightening agents include sodium silicate and magnesium aluminum silicate. Suitable botanicals include rosa damascena (rose) flower extract, cucumis sativus (cucumber) fruit extract, and glycyrrhiza glabra (licorice) root extract. Suitable film formers include pullulan. Suitable peptide compounds include Acetyl Hexapeptide—8, Caprylyl Glycol, Water Extract. Suitable thickeners include xanthan gum. Suitable preservatives include phenoxyethanol and ethylhexylglycerin.

Some embodiments of the serum may comprise sodium silicate, glycerin extract, pullulan, propylene glycol, magnesium aluminum silicate, and xanthan gum, wherein variations in any of these materials may lead to a negative effect in product performance and product esthetics, such as an increase in whitening, a reduced lifting effect, and a heavier, stickier skin feel, which causes the product to become powdery on the skin. The serum may further comprise water, rose lower extract, cucumber fruit extract, licorice root extract, phenoxyethanol, ethylhexylglycerin, and Acetyl Hexapeptide—8, Caprylyl Glycol, Water Extract.

A particular embodiment of the serum may comprise from about 5 to about 20% by volume (vol %) sodium silicate; about 5 to about 25 vol % rose flower extract; about 5 to about 10 vol % propylene glycol; less than about 10 vol % magnesium aluminum silicate; about 1 to about 5 vol % glycerin; less than about 1 vol % pullulan; about 5 to about 15 vol % acetyl hexapeptide—8, caprylyl glycol, water extract; less than about 2 vol % cucumber fruit extract; less than about 2 vol % licorice root extract; less than about 5 vol % xanthan gum; less than about 1 vol % phenoxyethanol; less than about 1 vol % ethylhexylglycerin; and water in an amount to equal a total of 100 vol %.

When it comes to the cucumber and licorice extracts, glycerin may be used as the primary solvent for the production of the extracts, because of additional skin benefits it may impart. Moreover, the combination of both cucumber and licorice may offer a soothing effect alongside the humectant (moisturizing) effects of glycerin. Thus, the use of these extracts may offset dryness frequently associated with the presence of high levels of silicates and may help eliminate the typical white demarcation lines that are typically associated with products of this nature. The amount of glycerin (about 1 to about 5 vol %) may be ideal. If too little glycerin is used, the moisturizing benefits may not be seen, and the silicate film may turn white. Conversely, if too much is used, the hygroscopic properties of the glycerin may become too great such that they overwhelm the silicates and prevent the silicates from drying fulling, thus reducing or completely removing the lifting and tightening effect.

Pullulan is a naturally occurring, low viscosity polysaccharide that has good adhesive properties on the skin when dissolved at less than 1 vol % in water. These properties allow pullulan to be used for creating luxuriant smoothness for skin care products off-setting dryness and leaving a natural silicone-like after feel on the skin. With its film-forming ability, pullulan blended with the combined silicate levels as described above, may assist the adhesiveness of the serum to the skin, enhancing the lifting effect of the product on wrinkles.

Xanthan gum is a natural gum thickener that may provide the serum with a pleasant skin feel and rheology. If used alone, the xanthan gum may only be able to provide a mobile gel-type product with a gloopy appearance. When used at levels of more than about 5 vol %, the xanthan gum may not provide a firm gel/serum type rheology and may, instead, result in a sticky, unpleasant skin eel. When used in an amount of less than about 5 vol %, the combination and ratio of xanthan gum to magnesium aluminum silicate may be the key to providing a firm gel with a good pick up, excellent skin feel, and minimal level of whitening.

Propylene glycol may be used primarily as a dispersant for the xanthan gum, because it may help to hydrate it faster than any other dispersant. Propylene glycol is also hygroscopic in nature, which means it may work to prevent the sodium silicate film from drying out completely and becoming white. Propylene glycol is less sticky than glycerin and, thus, does not result in a potentially sticky, heavy feel from using glycerin alone. Thus, the use of both propylene glycol and glycerin may be beneficial in the serum of the present disclosure.

Magnesium aluminum silicate is a mineral thickener that has sheer thinning properties. Using magnesium aluminum silicate in the serum of the present disclosure may give the serum a firmness while not feeling too heavy. The sheer thinning may help with the light skin feel when the product is applied, as the viscosity quickly reduces. Used alone, magnesium aluminum silicate may not provide an ideal thickness or rheology to the product, due to its mineral composition. Thus, if magnesium aluminum silicate is used as the sole thickener, the serum may become an extremely firm gel that is almost set solid. Additionally, using magnesium aluminum silicate in an amount of more than about 10 vol % may leave a whitening effect upon drying, as it is an insoluble mineral.

Sodium silicate may provide instant lifting and tightening properties. As the serum dries on the skin, it may leave a film of sodium silicate. The film may tighten the skin, thus causing the lifting effect. When the sodium silicate dries completely on the skin, it is traditionally known to leave a visible white demarcation where it has been applied, which is more apparent in the fine lines and wrinkles, because more product is deposited in these areas. When sodium silicate is included in the serum in an amount of from about 5 to about 20 vol %, the serum may dry clear on the skin. When less than about 5 vol % sodium silicate is used, the lifting performance may be diminished. When more than about 20 vol % sodium silicate is used, the appearance of white marks may increase as the serum drives.

The serum of the present disclosure may be made using the following method. If the method is varied, the performance of the resulting product may suffer.

To manufacture the serum of the present disclosure, the production may be carried out in a stainless steel manufacturing vessel fitted with stirrers, such as contra-rotating stirrers, and a homogenizer, such as a bottom mounted homogenizer. Water, such as deionized water, cucumber fruit organic glycerine extract, licorice root organic glycerine extract, ethylhexylglycerin, phenoxyethanol, rosewater organic extract, and pullulan may be added to the main vessel. In a suitable separate container, propylene glycol and xanthan gum may be mixed together for a time period of, for example, about 15 minutes to create a pre-mix. Such mixing time may ensure the correct level of hydration.

In the main vessel, the homogenizer may be turned on to a high speed, such as a speed of about 2800 rpm. At this time, the stirrer blades should remain off to prevent creating an airflow around the inside of the vessel that disperses the magnesium aluminum silicate. If the magnesium aluminum silicate contacts a damp surface, it will hydrate and stick to that surface. It will also stick to, and accumulate on, the stirrer blades. Once magnesium aluminum silicate has hydrated in this way, it can create small lumps of non-hydrated powder within a hydrated shell, which can then be difficult to disperse. Additionally, the magnesium aluminum silicate can hydrate fully and form a thick gel due to the low water levels it is hydrated in. This gel can then be broken down into small gel particles by the friction of the side sweep fixings on the mixer as they contact the vessel surface. These particles can then fall into, or be drawn into the batch as the batch volume increases in the vessel. It is then difficult to remove the particles from the batch. As such, and as mentioned above, the stirrer blades should remain off during this homogenization stage.

While the homogenizer is on its high speed, magnesium aluminum silicate may be sprinkled into the vortex created by the homogenizer. Before proceeding further, the process may include confirming that there are no lumps that have formed from the addition of the magnesium aluminum silicate. This may be done by lifting the blades from the vessel to check for lumps. Once it has been confirmed that there are no lumps, the stirrer blades may be turned on and homogenization and stirring may continue for about 15 minutes. During this time, the homogenizer may be operating as fast as possible without allowing the vortex to draw air into the bulk. In some embodiments, the homogenizer speed may be about 2800 rpm to ensure sufficient mixing without aeration of the bulk.

Once the homogenization/stirring has run for about 15 minutes after the addition of the magnesium aluminum silicate, the pre-mix may be added into the main vessel. Homogenization and stirring may continue for about 15 minutes until the mixture is smooth and free of lumps. As with the magnesium aluminum silicate, it may be advantageous to add the pre-mix directly into the vortex in the main vessel. Although the stirrer being on may make this slightly more difficult, the stirrer should remain running to prevent the premix from settling to the bottom of the main vessel and hydrating as one lump at the bottom of the mix. After about 2 minutes, the homogenizer may be turned off, because as the batch thickens the homogenizer may not add any benefit to the mixing, and it may increase the likelihood that air will be drawn in, which is then difficult to remove. While the homogenizer may be turned off, the stirrer blades may be kept on a high speed to keep the batch more mobile by preventing the magnesium aluminum silicate from thickening. Because magnesium aluminum silicate is a sheer thinning ingredient, the rapid agitation may help keep it mobile. The high stirrer speed may also result in fully dispersing and hydrating the xanthan gum. If the mixer is operating at too slow of a speed, the xanthan gum may take far longer to disperse, which may be evident by small, gel-like lumps of hydrating xanthan gum noticeable in the mixture. The fast moving of the stirrer blades may break the lumps down and disperse them fully and more rapidly, ultimately leading to a smoother finished product. In embodiments, suitable stirrer speeds may be from about 30 to about 35 rpm.

While stirring continues in the main vessel, sodium silicate may be added to the main vessel. The sodium silicate may be added slowly with fast mixing at a stirrer speed of from about 30 to about 35 rpm. Stirring may continue for about 15 to 20 minutes until the mixture is thoroughly mixed. Maintaining a high stirrer speed may ensure that the sodium silicate is dispersed as rapidly as possible to prevent the formation of large drops or volumes of the liquid. When sodium silicate is added too quickly or if the batch is not being mixed quickly enough, then the sodium silicate has the tendency to precipitate/agglomerate, which may appear as a stringy white substance. Once a sodium silicate agglomeration is formed, it is insoluble and cannot be redissolved and, thus, formation of such agglomeration may make the batch aesthetically a reject. The formation of the agglomeration may also negatively affect the performance of the final product if the material could not be filtered out.

Once the above step is completed, a sample of the product may be analyzed by quality assurance to check the product performance on the skin. For the product to pass quality control standards, use of the serum may not create a visible white demarcation line, and the product may feel hydrating on the skin, wherein the texture may have a consistency similar to petroleum jelly.

Upon receiving approval from quality assurance, a peptide based active ingredient, such as an argireline solution, may be added to the main vessel, which may be stirred for about 30 minutes until the mixture is thoroughly mixed and uniform, creating the final serum. The argireline solution may have a Botox-like effect on the skin. The argireline solution may be added slowly to the bulk with fast stirring (about 30 to about 35 rpm) to ensure that the argireline solution is quickly dispersed to prevent negative reactions. The final serum may again be sent to quality assurance for approval. While testing of the product is occurring, it may be beneficial to leave the mixing blades running at a speed of about 15 rpm to keep the serum as mobile as possible, which will ensure easier removal from the vessel for packaging.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A serum for improving the youthful appearance of skin, the serum comprising:
sodium silicate;
glycerin extract;
pullulan;
propylene glycol;
magnesium aluminum silicate; and
xanthan gum.

2. The serum of claim 1, further comprising:
water;
rosa damascena flower extract;
acetyl hexapeptide—8, caprylyl glycol, water extract;
cucumis sativus fruit extract;
glycyrrhiza glabra root extract;
phenoxyethanol; and
ethylhexylglycerin.

3. The serum of claim 2, wherein the serum comprises, based on a total of 100% by volume (vol %):
about 5 to about 20 vol % sodium silicate;
about 5 to about 25 vol % rosa damascena flower extract;
about 5 to about 10 vol % propylene glycol;
less than about 10 vol % magnesium aluminum silicate;
about 1 to about 5 vol % glycerin;
less than about 1 vol % pullulan;
about 5 to about 15 vol % acetyl hexapeptide—8, caprylyl glycol, water extract;
less than about 2 vol % cucumis sativus fruit extract;
less than about 2 vol % glycyrrhiza glabra root extract;
less than about 5 vol % xanthan gum;
less than about 1 vol % phenoxyethanol;
less than about 1 vol % ethylhexylglycerin; and water.

4. A serum for improving the youthful appearance of skin, the serum comprising:
a solvent;
a tightening agent;
a botanical;
a fragrance;
a humectant;
a film former;
a peptide compound;
a thickener; and
a preservative.

5. The serum of claim 4, wherein:
the solvent is at least one member selected from the group consisting of water and propylene glycol;
the tightening agent is at least one member selected from the group consisting of sodium silicate and magnesium aluminum silicate;
the botanical is at least one member selected from the group consisting of rosa damascena flower extract, cucumis sativus fruit extract, and glycyrrhiza glabra root extract;
the fragrance is rosa damascena flower extract;
the humectant is glycerin;
the film former is pullulan;
the peptide compound is acetyl hexapeptide—8, caprylyl glycol, water extract;
the thickener is at least one member selected from the group consisting of magnesium aluminum silicate and xanthan gum; and
the preservative is at least one member selected from the group consisting of phenoxyethanol and ethylhexylglycerin.

6. A method of manufacturing a skin serum for improving a youthful appearance of skin, the method comprising:
adding water, cucumber fruit organic glycerin extract, licorice root organic glycerin extract, ethylhexylglycerin, phenoxyethanol, rosewater organic extract, and pullulan to a main vessel, creating a first mixture;
mixing together propylene glycol and xanthan gum in a separate container, creating a premix;
homogenizing the first mixture without turning on stirrer blades in the main vessel;
adding magnesium aluminum silicate into a vortex created by the homogenizer in the main vessel;
once the magnesium aluminum silicate is added into the main vessel, turning on the stirrer blades in the main vessel;
adding the premix into the main vessel while continuing to stir and homogenize, creating an intermediate mixture;
adding sodium silicate to the intermediate mixture in the main vessel while stirring and homogenizing; and
adding an argireline solution to the main vessel while stirring, wherein the argireline solution comprises acetyl hexapeptide—8, caprylyl glycol, water extract,
wherein the serum comprises, based on a total of 100% by volume (vol %):
about 5 to about 20 vol % sodium silicate;
about 5 to about 25 vol % rosa damascena flower extract;
about 5 to about 10 vol % propylene glycol;
less than about 10 vol % magnesium aluminum silicate;
about 1 to about 5 vol % glycerin;
less than about 1 vol % pullulan;
about 5 to about 15 vol % acetyl hexapeptide—8, caprylyl glycol, water extract;
less than about 2 vol % cucumis sativus fruit extract;
less than about 2 vol % glycyrrhiza glabra root extract;
less than about 5 vol % xanthan gum;
less than about 1 vol % phenoxyethanol;
less than about 1 vol % ethylhexylglycerin; and
water.
